(19) United States
(12) Patent Application Publication
(10) Pub. No.: US 2016/0367397 A1
(43) Pub. Date: Dec. 22, 2016

(54) MULTI-FUNCTION CAPSULORHEXIS GUIDE

(71) Applicant: Katalyst Surgical, LLC, Chesterfield, MO (US)

(72) Inventors: Michael S. Korenfeld, Wildwood, MO (US); Gregg D. Scheller, Wildwood, MO (US); Johanna L. Bryan, Lincoln, NE (US)

(73) Assignee: KATALYST SURGICAL, LLC, Chesterfield, MO (US)

(21) Appl. No.: 14/745,900

(22) Filed: Jun. 22, 2015

(51) Int. Cl. A61F 9/007; A61B 17/02

(52) U.S. Cl. CPC A61F 9/007; A61B 17/0231

(57) ABSTRACT

A multi-function capsulorhexis guide may include an outer rim, an inner rim, an anterior surface, a capsulorhexis guide, and an iris contact angle. The outer rim may include an outer rim medial edge, an outer rim lateral edge, and an outer rim thickness. The inner rim may comprise an inner rim medial edge, an inner rim lateral edge, and an inner rim thickness. The capsulorhexis guide may be configured to guide a tearing of a lens capsule. The anterior surface may include an adhesion geometry configured to temporarily fix the multi-function capsulorhexis guide to a portion of the lens capsule. The iris contact angle may be configured to retract an iris.

### CROSS-REFERENCE TO RELATED APPLICATIONS

This Application claims the benefit of U.S. Provisional Application No. 62/036,389, filed Aug. 12, 2014.

### FIELD OF THE INVENTION

The present disclosure relates to a medical device, and, more particularly, to an ophthalmic medical device.

### BACKGROUND OF THE INVENTION

Capsulorhexis is a surgical removal of a portion of a lens capsule. Removing the portion of the lens capsule creates an opening in the lens capsule that enables a surgeon to remove a cataract and insert an artificial lens. Separating the portion of the lens capsule from the remaining lens capsule may be accomplished using laser, a radio frequency probe, a needle, a forceps, etc. A common method of separating the portion of the lens capsule from the remaining lens capsule is continuous curvilinear capsulorhexis (“CCC”) wherein a surgeon uses a series of incisions and tears to form an opening in the lens capsule. The geometry and dimensions of the opening in the lens capsule must be precise to prevent unintended tearing of the lens capsule. Achieving precise geometry and dimensions of an opening in a lens capsule may be difficult, e.g., due to a small pupil diameter, zonular weakness, etc. Pupil diameter is typically controlled pharmacologically; however, in some cases a surgeon is unable to successfully accomplish a pharmacological retraction of an iris and the iris must be retracted mechanically to enlarge a pupil diameter. Moreover, it may be difficult for a surgeon to accomplish precise incisions when performing CCC because a surface of the lens capsule may slightly deform as force is applied to the surface during an attempted incision. The initial deformation in the surface of the lens capsule before an instrument pierces the lens capsule may cause undesirable incision geometry and subsequent tearing of the lens capsule may be difficult for a surgeon to control.

### BRIEF SUMMARY OF THE INVENTION

A multi-function capsulorhexis guide is presented. Illustratively, a multi-function capsulorhexis guide may comprise an outer rim, an inner rim, an anterior surface, a capsulorhexis guide, and an iris contact angle. In one or more embodiments, the outer rim may comprise an outer rim medial edge, an outer rim lateral edge, and an outer rim thickness. Illustratively, the inner rim may comprise an inner rim medial edge, an inner rim lateral edge, and an inner rim thickness. In one or more embodiments, the capsulorhexis guide may be configured to guide a tearing of a lens capsule. Illustratively, the anterior surface may comprise an adhesion geometry configured to temporarily fix the multi-function capsulorhexis guide to a portion of the lens capsule. In one or more embodiments, the iris contact angle may be configured to retract an iris.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, 1C, and 1D are schematic diagrams illustrating a multi-function capsulorhexis guide;

FIG. 2 is a schematic diagram illustrating a retracted iris;

FIG. 3 is a schematic diagram illustrating a capsulorhexis.

### DETAILED DESCRIPTION OF AN ILLUSTRATIVE EMBODIMENT

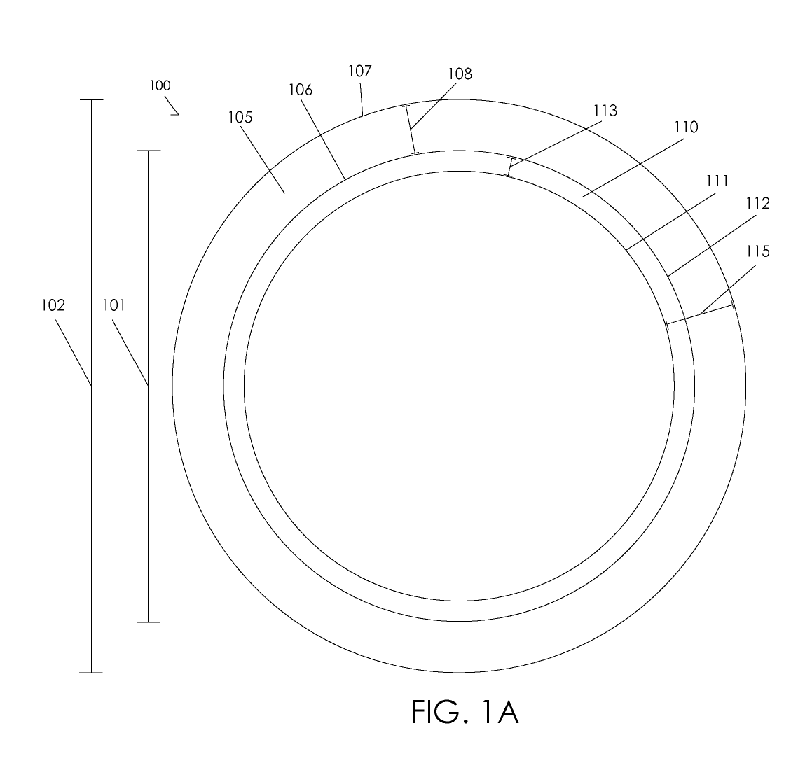

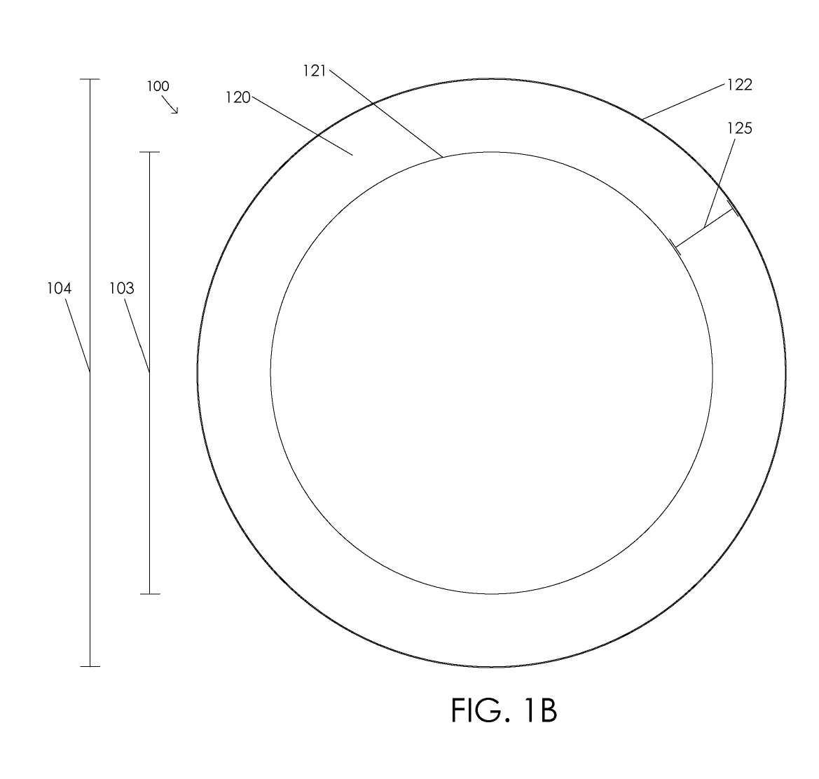

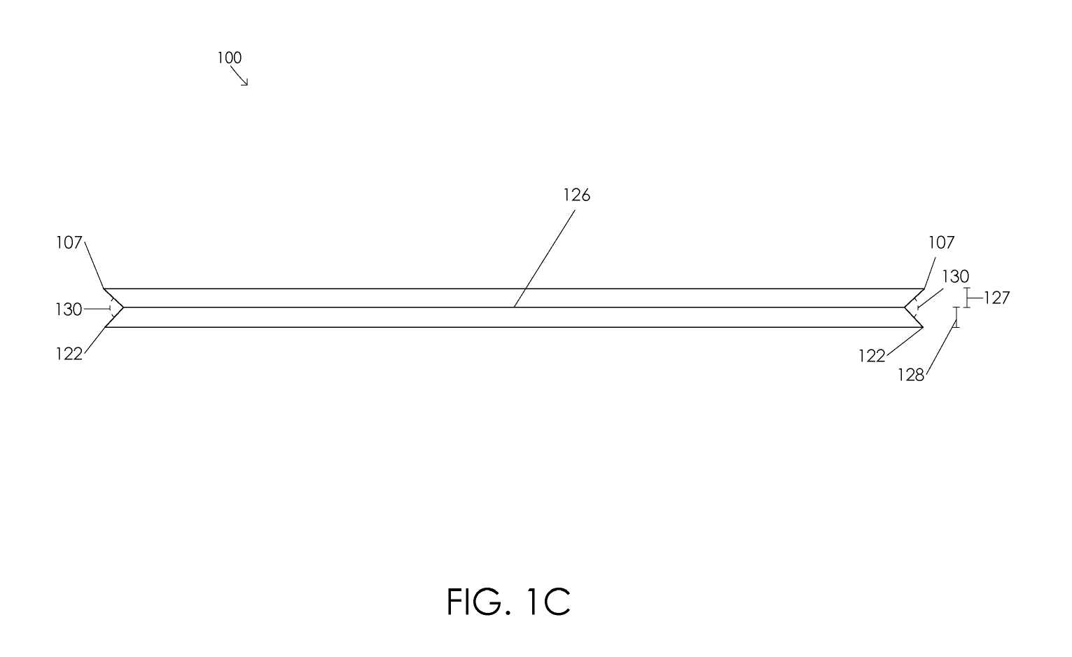

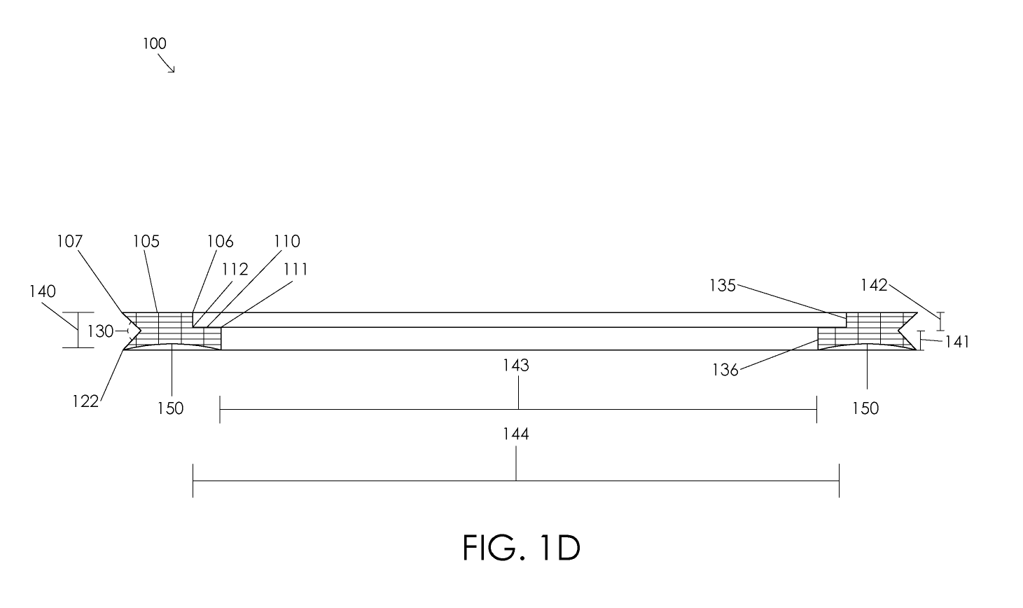

FIGS. 1A, 1B, 1C, and 1D are schematic diagrams illustrating a multi-function capsulorhexis guide 100. FIG. 1A illustrates a top view of a multi-function capsulorhexis guide 100. In one or more embodiments, multi-function capsulorhexis guide 100 may comprise an outer rim 105, an inner rim 110, and a multi-function capsulorhexis guide anterior thickness 115. Illustratively, multi-function capsulorhexis guide anterior thickness 115 may be a distance in a range of 0.5 to 1.0 millimeters, e.g., multi-function capsulorhexis guide anterior thickness 115 may be a distance of 0.875 millimeters. In one or more embodiments, multi-function capsulorhexis guide anterior thickness 115 may be a distance of less than 0.5 millimeters or greater than 1.0 millimeters. Illustratively, outer rim 105 may comprise an outer rim inner diameter 101, an outer rim outer diameter 102, an outer rim medial edge 106, an outer rim lateral edge 107, and an outer rim thickness 108. In one or more embodiments, outer rim inner diameter 101 may be a distance in a range of 5.0 to 7.0 millimeters, e.g., outer rim inner diameter 101 may be a distance of 5.75 millimeters. Illustratively, outer rim inner diameter 101 may be a distance of less than 5.0 millimeters or greater than 7.0 millimeters. In one or more embodiments, outer rim outer diameter 102 may be a distance in a range of 6.0 to 8.0 millimeters, e.g., outer rim outer diameter 102 may be a distance of 7.0 millimeters. Illustratively, outer rim outer diameter 102 may be a distance of less than 6.0 millimeters or greater than 8.0 millimeters. In one or more embodiments, outer rim thickness 108 may be a distance in a range of 0.5 to 1.2 millimeters, e.g., outer rim thickness 108 may be a distance of 0.625 millimeters. Illustratively, outer rim thickness 108 may be a distance of less than 0.5 millimeters or greater than 1.2 millimeters. In one or more embodiments, inner rim 110 may comprise an inner rim medial edge 111, an inner rim lateral edge 112, and an inner rim thickness 113. Illustratively, inner rim thickness 113 may be a distance in a range of 0.1 to 0.5 millimeters, e.g., inner rim thickness 113 may be a distance of 0.25 millimeters. In one or more embodiments, inner rim thickness 113 may be a distance of less than 0.1 millimeters or greater than 0.5 millimeters.

FIG. 1B illustrates a bottom view of a multi-function capsulorhexis guide 100. Illustratively, multi-function capsulorhexis guide 100 may comprise a posterior surface 120, a posterior surface inner diameter 103, and a posterior surface outer diameter 104. In one or more embodiments, posterior surface inner diameter 103 may be a distance in a range of 4.5 to 6.5 millimeters, e.g., posterior surface inner diameter 103 may be a distance of 5.25 millimeters. Illustratively, posterior surface inner diameter 103 may be a distance of less than 4.5 millimeters or greater than 6.5 millimeters. In one or more embodiments, posterior surface outer diameter 104 may be a distance in a range of 6.0 to 9.0 millimeters, e.g., poster surface outer diameter 104 may be a distance of 7.0 millimeters. Illustratively, posterior surface outer diameter 104 may be a distance of less than 6.0 millimeters or greater than 9.0 millimeters. In one or more embodiments, posterior surface 120 may comprise a posterior surface medial edge 121, a posterior surface lateral edge 122, and a posterior surface thickness 125. Illustratively, posterior surface thickness 125 may be a distance in a range of 0.25 to 1.5 millimeters, e.g., posterior surface thickness 125 may be a distance of 0.875 millimeters. In one or more embodiments, posterior surface thickness 125 may be a distance of less than 0.25 millimeters or greater than 1.5 millimeters.

FIG. 1C illustrates a side view of a multi-function capsulorhexis guide 100. In one or more embodiments, multi-function capsulorhexis guide 100 may comprise an iris inner diameter interface 126, a dorsal height 127, a ventral depth 128, and an iris contact angle 130. Illustratively, iris inner diameter interface 126 may be configured to interface with an iris 210, e.g., iris inner diameter interface 126 may be configured to interface with an anterior boarder layer, a stroma, and an iris pigment epithelium. For example, iris inner diameter interface 126 may be configured to interface with an iris inner diameter 211. In one or more embodiments, dorsal height 127 may be a distance in a range of 0.05 to 0.25 millimeters, e.g., dorsal height 127 may be a distance of 0.165 millimeters. Illustratively, dorsal height 127 may be a distance of less than 0.05 millimeters or greater than 0.25 millimeters. In one or more embodiments, ventral depth 128 may be a distance in a range of 0.05 to 0.25 millimeters, e.g., ventral depth 128 may be a distance of 0.165 millimeters. Illustratively, ventral depth 128 may be a distance of less than 0.05 millimeters or greater than 0.25 millimeters. In one or more embodiments, iris contact angle 130 may be configured to interface with an iris 210, e.g., iris contact angle 130 may be configured to interface with an iris 210 without causing trauma to an anterior boarder layer, a stroma, and an iris pigment epithelium. Illustratively, iris contact angle 130 may be an angle in a range of 45.0 to 120.0 degrees, e.g., iris contact angle 130 may be an angle of 90.0 degrees. In one or more embodiments, iris contact angle 130 may be an angle of less than 45.0 degrees or greater than 120.0 degrees. Illustratively, multi-function capsulorhexis guide 100 may comprise a first outer lip extending between outer rim lateral edge 107 and iris inner diameter interface 126. In one or more embodiments, the first outer lip may have a length in a range of 0.1 to 0.3 millimeters, e.g., the first outer lip may have a length of 0.233 millimeters. Illustratively, the first outer lip may have a length of less than 0.1 millimeters or greater than 0.3 millimeters. In one or more embodiments, multi-function capsulorhexis guide 100 may comprise a second outer lip extending between posterior surface lateral edge 122 and iris inner diameter interface 126. Illustratively, the second outer lip may have a length in a range of 0.1 to 0.3 millimeters, e.g., the second outer lip may have a length of 0.233 millimeters. In one or more embodiments, the second outer lip may have a length of less than 0.1 millimeters or greater than 0.3 millimeters.

FIG. 1D illustrates a cross-sectional view of a multi-function capsulorhexis guide 100. Illustratively, multi-function capsulorhexis guide 100 may comprise an outer rim interface 135, a capsulorhexis guide 136, a multi-function capsulorhexis guide height 140, an inner rim height 141, and outer rim height 142, an inner rim inner diameter 143, an inner rim outer diameter 144, and an adhesion geometry 150. In one or more embodiments, multi-function capsulorhexis guide height 140 may be a distance in a range of 0.2 to 0.6 millimeters, e.g., multi-function capsulorhexis guide height 140 may be a distance of 0.33 millimeters. Illustratively, multi-function capsulorhexis guide height 140 may be a distance of less than 0.2 millimeters or greater than 0.6 millimeters. In one or more embodiments, inner rim height 141 may be a distance in a range of 0.05 to 0.2 millimeters, e.g., inner rim height 141 may be a distance of 0.13 millimeters. Illustratively, inner rim height 141 may be a distance of less than 0.05 millimeters or greater than 0.2 millimeters. In one or more embodiments, outer rim height 142 may be a distance in a range of 0.1 to 0.3 millimeters, e.g., outer rim height 142 may be a distance of 0.2 millimeters. Illustratively, outer rim height 142 may be a distance of less than 0.1 millimeters or greater than 0.3 millimeters. In one or more embodiments, inner rim inner diameter 143 may be a distance in a range of 4.0 to 7.0 millimeters, e.g., inner rim inner diameter 143 may be a distance of 5.25 millimeters. Illustratively, inner rim inner diameter 143 may be a distance of less than 4.0 millimeters or greater than 7.0 millimeters. In one or more embodiments, inner rim outer diameter 144 may be a distance in a range of 4.5 to 7.5 millimeters, e.g., inner rim outer diameter 144 may be a distance of 5.75 millimeters. Illustratively, inner rim outer diameter 144 may be a distance of less than 4.5 millimeters or greater than 7.5 millimeters.

Illustratively, adhesion geometry 150 may be configured to temporarily fix a portion of multifunction capsulorhexis guide 100 to a portion of a lens capsule 240, e.g., adhesion geometry 150 may be configured to temporarily fix posterior surface 120 to a portion of a lens capsule outer surface 241. In one or more embodiments, adhesion geometry 150 may be configured to facilitate an adhesion of multi-function capsulorhexis guide 100 and a portion of a lens capsule 240, e.g., adhesion geometry 150 may be configured to facilitate suction by creating a pressure differential between a fluid contained within adhesion geometry 150 and an adjacent fluid on a portion of a lens capsule 240. Illustratively, adhesion geometry 150 may comprise a micro-scale etched pattern configured to facilitate an adhesion of multi-function capsulorhexis guide 100 and a portion of a lens capsule 240, e.g., adhesion geometry 150 may be configured to facilitate van der Waals forces between a micro-scale etched pattern of adhesion geometry 150 an a portion of a lens capsule 240. In one or more embodiments, adhesion geometry 150 may be configured facilitate an adhesion of multi-function capsulorhexis guide 100 and a portion of a lens capsule 240, e.g., adhesion geometry 150 may house an adhesive configured to fix a portion of multi-function capsulorhexis guide 100 to a portion of a lens capsule 240. Illustratively, adhesion geometry 150 may comprise a convex geometry relative to posterior surface 120. In one or more embodiments, adhesion geometry 150 may have a convex peak in a range of 0.001 to 0.1 millimeters, e.g., adhesion geometry 150 may have a convex peak of 0.05 millimeters. Illustratively, adhesion geometry 150 may have a convex peak of less than 0.001 millimeters or greater than 0.1 millimeters.

In one or more embodiments, adhesion geometry 150 may be configured to house a recessed blade, e.g., adhesion geometry 150 may house a recessed blade configured to incise a portion of lens capsule 240. Illustratively, multi-function capsulorhexis guide 100 may comprise a recessed blade having a recessed blade distal end and a recessed blade proximal end wherein the recessed blade is disposed within adhesion geometry 150, e.g., the recessed blade proximal end may abut a convex peak of adhesion geometry 150 and the recessed blade distal end may be disposed inferior of posterior surface 120. For example, the recessed blade distal end may not extend beyond posterior surface 120 when multi-function capsulorhexis guide 100 is in equilibrium. In one or more embodiments, multi-function capsulorhexis guide 100 may comprise a recessed blade wherein an application of a force to a portion of multi-function capsulorhexis guide 100 may be configured to extend a distal end of the recessed blade a distance beyond posterior surface 120, e.g., an application of a force to outer rim 105 may be configured to extend the distal end of the recessed blade a distance beyond posterior surface 120. Illustratively, an extension of a distal end of a recessed blade a distance beyond posterior surface 120 may be configured to incise a portion of lens capsule 240.

In one or more embodiments, multi-function capsulorhexis guide 100 may be manufactured from any suitable material, e.g., polymers, metals, metal alloys, etc., or from any combination of suitable materials. Illustratively, multi-function capsulorhexis guide 100 may be manufactured from a material having a hardness in a range of 50 durometer Shore A to 80 durometer Shore A, e.g., multi-function capsulorhexis guide 100 may be manufactured from a material having a hardness of 70 durometer Shore A. In one or more embodiments, multi-function capsulorhexis guide 100 may be manufactured from a material having a hardness less than 50 durometer Shore A or greater than 80 durometer Shore A. Illustratively, multi-function capsulorhexis guide 100 may be manufactured from a material having a compression set after 70 hours at 150.0 degrees Celsius in a range of 20.0 to 30.0 percent, e.g., multi-function capsulorhexis guide 100 may be manufactured from a material having a compression set after 70 hours at 150.0 degrees Celsius of 25.0 percent. In one or more embodiments, multi-function capsulorhexis guide 100 may be manufactured from a material having a compression set after 70 hours at 150.0 degrees Celsius of less than 20.0 percent or greater than 30.0 percent. Illustratively, multi-function capsulorhexis guide 100 may be manufactured from a material having a tensile strength in a range of 6.0 to 9.0 MPa, e.g., multi-function capsulorhexis guide 100 may be manufactured from a material having a tensile strength of 7.8 MPa. In one or more embodiments, multi-function capsulorhexis guide 100 may be manufactured from a material having a tensile strength of less than 6.0 MPa or greater than 9.0 MPa. Illustratively, multi-function capsulorhexis guide 100 may be manufactured from a material having an elongation in a range of 180.0 to 220.0 percent, e.g., multi-function capsulorhexis guide 100 may be manufactured from a material having an elongation of 200.0 percent. In one or more embodiments, multi-function capsulorhexis guide 100 may be manufactured from a material having an elongation of less than 180.0 percent or greater than 220.0 percent. Illustratively, multi-function capsulorhexis guide 100 may be manufactured from a material having a tear strength in a range of 12.0 to 19.0 kN/m, e.g., multi-function capsulorhexis guide 100 may be manufactured from a material having a tear strength of 15.5 kN/m. In one or more embodiments, multi-function capsulorhexis guide 100 may be manufactured from a material having a tear strength of less than 12.0 kN/m or greater than 19.0 kN/m.

Illustratively, multi-function capsulorhexis guide 100 may be manufactured from a material suitable for sterilization by ethylene oxide, e.g., multi-function capsulorhexis guide 100 may be a single-use, disposable medical device. In one or more embodiments, multi-function capsulorhexis guide 100 may be manufactured from a material suitable for sterilization by a medical autoclave, e.g., may be a multi-use, reusable medical device. Illustratively, multi-function capsulorhexis guide 100 may be manufactured from a material configured to withstand exposure to temperatures, pressures, and ambient conditions present in a medical autoclave without degradation. In one or more embodiments, multi-function capsulorhexis guide 100 may be configured to function normally after exposure in a temperature 250° F. for 15 minutes at an atmospheric pressure of 15 psi. Illustratively, multi-function capsulorhexis guide 100 may be configured to be used in a surgical procedure and then sterilized by a medical autoclave at least three times. In one or more embodiments, multi-function capsulorhexis guide 100 may be sterilized in a medical autoclave and then multi-function capsulorhexis guide 100 may be used in a first surgical procedure. Illustratively, multi-function capsulorhexis guide 100 may be sterilized in a medical autoclave after use in the first surgical procedure and then multi-function capsulorhexis guide 100 may be used in a second surgical procedure. In one or more embodiments, multi-function capsulorhexis guide 100 may be sterilized in a medical autoclave after use in the second surgical procedure and then multi-function capsulorhexis guide 100 may be used in a third surgical procedure.

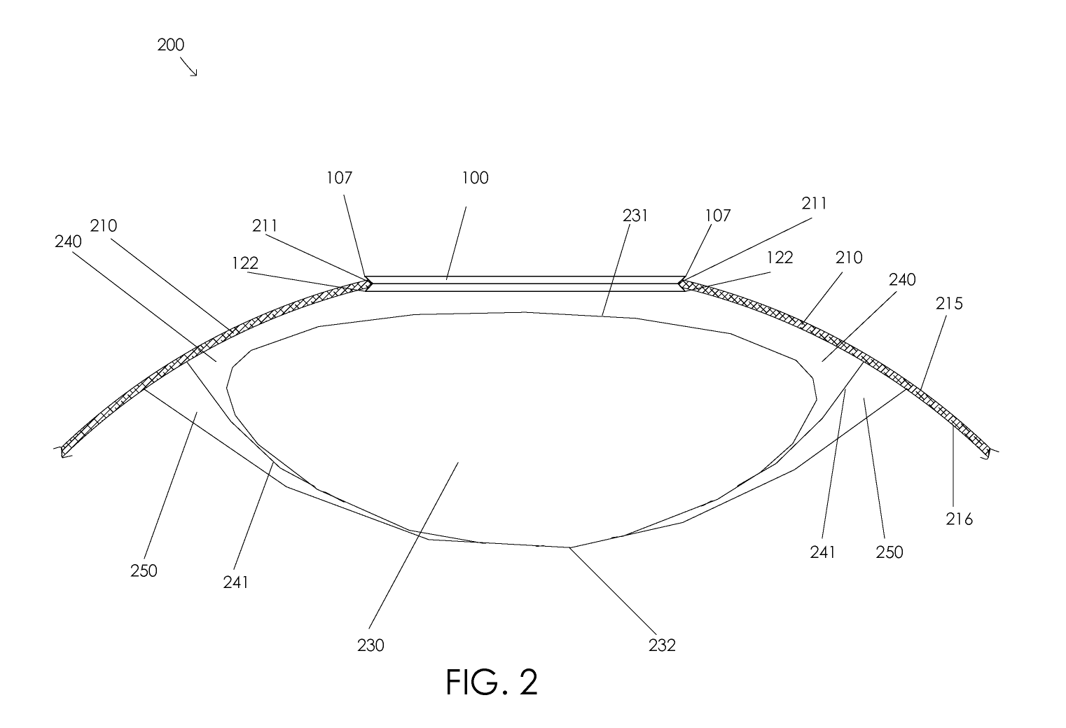

FIG. 2 is a schematic diagram illustrating a retracted iris 200. In one or more embodiments, retracted iris 200 may comprise a multi-function capsulorhexis guide 100, an iris 210, a lens 230, and a lens capsule 240. Illustratively, iris 210 may comprise an iris inner diameter 211, an iris anterior surface 215, and an iris posterior surface 216. In one or more embodiments, a surgeon may insert multi-function capsulorhexis guide 100 through an incision in a cornea wherein multi-function capsulorhexis guide 100 may be compressed and elongated during an insertion, e.g., multi-function capsulorhexis guide 100 may be compressed and elongated by a hypodermic tube of an inserter mechanism. For example, a surgeon may insert multi-function capsulorhexis guide 100 through an incision in a sclera 305 wherein multi-function capsulorhexis guide 100 may be compressed and elongated during an insertion. Illustratively, the surgeon may begin to insert multi-function capsulorhexis guide 100 into a pupil wherein multi-function capsulorhexis guide 100 may be gradually decompressed as multi-function capsulorhexis guide 100 ingresses the pupil. In one or more embodiments, a gradual decompression of a compressed and elongated multi-function capsulorhexis guide 100 may be configured to radially expand multi-function capsulorhexis guide 100. Illustratively, a radial expansion of multi-function capsulorhexis guide 100 may be configured to gradually retract iris 210, e.g., iris inner diameter interface 126 may gradually expand iris inner diameter 211 as multi-function capsulorhexis guide 100 radially expands. In one or more embodiments, a radial expansion of multi-function capsulorhexis guide 100 in a pupil may be configured to gradually expand the pupil. Illustratively, iris contact angle 130 may be configured to catch iris inner diameter 211 as multi-function capsulorhexis guide 100 radially expands. In one or more embodiments, iris contact angle 130 may be configured to ensure that outer rim lateral edge 107 is superior to iris anterior surface 215 as multi-function capsulorhexis guide 100 radially expands. Illustratively, iris contact angle 130 may be configured to ensure that posterior surface lateral edge 122 is inferior to iris posterior surface 216 as multi-function capsulorhexis guide 100 radially expands. In one or more embodiments, iris contact angle 130 may be configured to ensure that outer rim lateral edge 107 is superior to iris anterior surface 215 and posterior surface lateral edge 122 is inferior to iris posterior surface 216 as multi-function capsulorhexis guide 100 radially expands. Illustratively, iris contact angle 130 may be configured to guide iris inner diameter 211 towards iris inner diameter interface 126 as multi-function capsulorhexis guide 100 radially expands. In one or more embodiments, after a contact between iris inner diameter 211 and iris inner diameter interface 126, a radial expansion of multi-function capsulorhexis guide 100 may be configured to retract iris 210 until iris 210 comprises a retracted iris 200. Illustratively, the surgeon may manipulate multi-function capsulorhexis guide 100 as multi-function capsulorhexis guide 100 decompresses and radially expands in the pupil wherein iris inner diameter 211 is adjacent to iris inner diameter interface 126. In one or more embodiments, the surgeon may manipulate multi-function capsulorhexis guide 100 wherein outer rim lateral edge 107 may be disposed superior to iris anterior surface 215. Illustratively, the surgeon may manipulate multi-function capsulorhexis guide 100 wherein posterior surface lateral edge 122 may be disposed inferior to iris posterior surface 216. In one or more embodiments, iris 210 may comprise a retracted iris 200 when iris inner diameter 211 abuts iris inner diameter interface 126.

Illustratively, lens 230 may comprise a lens anterior end 231 and a lens posterior end 232. In one or more embodiments, lens 230 may be disposed within lens capsule 240. Illustratively, lens capsule 240 may comprise a lens capsule outer surface 241. In one or more embodiments, lens capsule 240 may be supported by zonules 250, e.g., zonules 250 may be adjacent to lens capsule outer surface 241. Illustratively, a portion of multi-function capsulorhexis guide 100 may be fixed to a portion of lens capsule 240 when iris 210 comprises a retracted iris 200, e.g., adhesion geometry 150 may be configured to fix posterior surface 120 to a portion of lens capsule 240. In one or more embodiments, adhesion geometry 150 may be configured to prevent multi-function capsulorhexis guide 100 from moving relative to lens capsule 240. Illustratively, adhesion geometry 150 may be configured to apply tension to a portion of lens capsule 240, e.g., adhesion geometry 150 may be configured to apply tension to a portion of lens capsule 240 disposed within inner rim inner diameter 143. In one or more embodiments, an application of tension to a portion of lens capsule 240 may be configured to reduce an amount of initial deformation of lens capsule outer surface 241 when a force is applied to lens capsule outer surface 241 during an attempted incision. Illustratively, a reduction of an amount of initial deformation of lens capsule outer surface 241 when a force is applied to lens capsule outer surface 241 during an attempted incision may be configured to improve an incision geometry. In one or more embodiments, an improvement in an incision geometry may be configured to reduce unintended tearing of lens capsule 240.

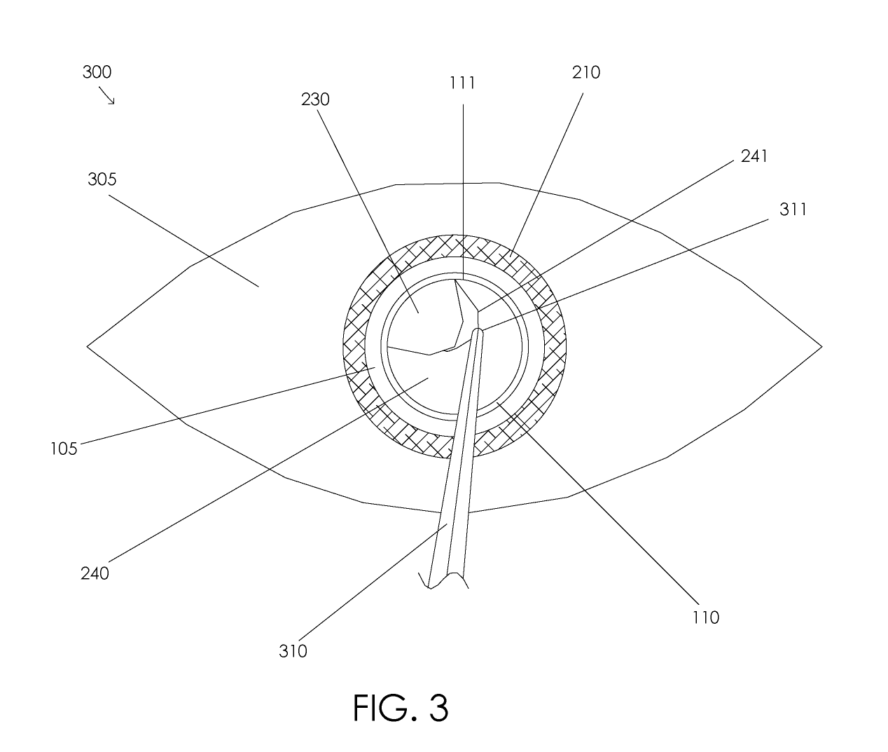

FIG. 3 is a schematic diagram illustrating a capsulorhexis 300. In one or more embodiments, a capsulorhexis 300 may comprise a forceps 310 having a forceps distal end 311. Illustratively, a surgeon may perform a capsulorhexis 300 by making an incision in lens capsule 240, e.g., a surgeon may make an incision in lens capsule 240 using a laser, a radio frequency probe, a needle, a forceps 310, etc. In one or more embodiments, a surgeon may perform a capsulorhexis 300 by making an incision in lens capsule 240 wherein the incision is adjacent to a portion of capsulorhexis guide 136. Illustratively, a surgeon may perform a capsulorhexis 300 by grasping a portion of lens capsule 240 with forceps 310 and tearing lens capsule 240 to create an opening in lens capsule 240, e.g., a surgeon may perform a capsulorhexis 300 by tearing lens capsule 240 along inner rim medial edge 111. In one more embodiments, a surgeon may perform a capsulorhexis 300 by grasping a portion of lens capsule outer surface 141 with forceps distal end 311 and tearing lens capsule outer surface 141 to create an opening in lens capsule 240, e.g., a surgeon may perform a capsulorhexis 300 by tearing lens capsule outer surface 241 along capsulorhexis guide 136. In one or more embodiments, performing a capsulorhexis 300 by tearing lens capsule 240 along capsulorhexis guide 136 may be configured to optimize a diameter of an opening in lens capsule 240, e.g., tearing lens capsule 240 along capsulorhexis guide 136 may be configured to create an opening in lens capsule 240 having a diameter of 5.25 millimeters. Illustratively, tearing lens capsule 240 along capsulorhexis guide 136 may be configured to create an opening in lens capsule 240 having a diameter equal to inner rim inner diameter 143. In one or more embodiments, tearing lens capsule 240 along capsulorhexis guide 136 may be configured to optimize a geometry of an opening in lens capsule 240, e.g., tearing lens capsule 240 along capsulorhexis guide 136 may be configured to create an opening in lens capsule 240 having a circular geometry.

Illustratively, a portion of multi-function capsulorhexis guide 100 may be configured to facilitate a tearing of lens capsule 240, e.g., a portion of multi-function capsulorhexis guide 100 may be configured to facilitate a tearing of lens capsule outer surface 241. In one or more embodiments, inner rim medial edge 111 may be configured to act as a pivot point for a portion of lens capsule outer surface 141, e.g., as a surgeon attempts to raise a portion of lens capsule outer surface 141 disposed within posterior surface inner diameter 103 using forceps 310, inner rim medial edge 111 may be configured to act as a pivot point for a portion of lens capsule outer surface 141 and facilitate a tearing of lens capsule outer surface 141 along capsulorhexis guide 136. Illustratively, inner rim medial edge 111 may be configured to provide a force to prevent a raising of a portion of lens capsule outer surface 141 and facilitate a tearing of lens capsule outer surface 141 along capsulorhexis guide 136. For example, inner rim medial edge 111 may be configured to cause a shearing force along inner rim medial edge 111 as a surgeon raises a portion of lens capsule outer surface 141 facilitating a tearing of lens capsule outer surface 141 along capsulorhexis guide 136. In one or more embodiments, adhesion geometry 150 may be configured to prevent multi-function capsulorhexis guide 100 from moving relative to lens capsule 240 during a capsulorhexis 300. Illustratively, adhesion geometry 150 may be configured to apply tension to a portion of lens capsule 240, e.g., adhesion geometry 150 may be configured to apply tension to a portion of lens capsule 240 disposed within inner rim inner diameter 143. In one or more embodiments, an application of tension to a portion of lens capsule 240 may be configured to facilitate a tearing of lens capsule outer surface 141 along inner rim medial edge 111.

Illustratively, multi-function capsulorhexis guide 100 may be configured to retract iris 210 during a capsulorhexis 300. In one or more embodiments, iris inner diameter 211 may abut iris inner diameter interface 126 during a capsulorhexis 300. Illustratively, multi-function capsulorhexis guide 100 may be configured to retract iris 210 and guide a capsulorhexis 300. In one or more embodiments, multi-function capsulorhexis guide 100 may be configured to provide a mechanical barrier to prevent a pharmacologically retracted iris 210 from contracting during a capsulorhexis 300. Illustratively, a surgeon may dilate a pupil pharmacologically and then begin a capsulorhexis 300. During the capsulorhexis 300, iris 210 may contract and prevent the surgeon from completing the capsulorhexis 300. In one or more embodiments, multi-function capsulorhexis guide 100 may be configured to prevent iris 210 from contracting during a capsulorhexis 300, e.g., multi-function capsulorhexis guide 100 may be configured to prevent a pharmacologically retracted iris 210 from contracting during a capsulorhexis 300.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of an ophthalmic medical device, the teachings of the present invention are equally suitable to any systems where the functionality may be employed. Therefore, it is the object of the appended claims to cover all such variations and modifications as come within the true spirit and scope of the invention.

What is claimed is:

1. An ophthalmic medical device comprising:
an outer rim having an outer rim medial edge, an outer rim lateral edge, and an outer rim thickness;
an inner rim having an inner rim medial edge, an inner rim lateral edge, and an inner rim thickness wherein the outer rim medial edge is adjacent to the inner rim lateral edge;
a capsulorhexis guide adjacent to the inner rim medial edge, the capsulorhexis guide configured to guide a tearing of a lens capsule;
a posterior surface having a posterior surface medial edge, a posterior surface lateral edge, and a posterior surface thickness;
an adhesion geometry of the posterior surface, the adhesion geometry configured to temporarily fix a portion of the posterior surface to an outer surface of the lens capsule;
an iris inner diameter interface; and
an iris contact angle.

2. The ophthalmic medical device of claim 1 wherein the outer rim thickness is a distance in a range of 0.5 to 1.2 millimeters.

3. The ophthalmic medical device of claim 1 wherein the inner rim thickness is a distance in a range of 0.1 to 0.5 millimeters.

4. The ophthalmic medical device of claim 1 wherein the poster surface thickness is a distance in a range of 0.25 to 1.5 millimeters.

5. The ophthalmic medical device of claim 1 wherein the outer rim has an inner diameter in a range of 5.0 to 7.0 millimeters.

6. The ophthalmic medical device of claim 1 wherein the outer rim has an outer diameter in a range of 6.0 to 8.0 millimeters.

7. The ophthalmic medical device of claim 1 wherein the inner rim has an inner diameter in a range of 4.0 to 7.0 millimeters.

8. The ophthalmic medical device of claim 1 wherein the inner rim has an outer diameter in a range of 4.5 to 7.5 millimeters.

9. The ophthalmic medical device of claim 1 wherein the posterior surface has an inner diameter in a range of 4.5 to 6.5 millimeters.

10. The ophthalmic medical device of claim 1 wherein the posterior surface has an outer diameter in a range of 6.0 to 9.0 millimeters.

11. The ophthalmic medical device of claim 1 wherein the iris contact angle is an angle in a range of 45.0 to 120.0 degrees.

12. The ophthalmic medical device of claim 1 wherein the iris inner diameter interface is configured to interface with an anterior boarder layer, a stroma, and an iris pigment epithelium.

13. The ophthalmic medical device of claim 1 wherein the iris inner diameter interface is configured to retract an iris.

14. The ophthalmic medical device of claim 1 further comprising:
a micro-scale etched pattern of the adhesion geometry.

15. The ophthalmic medical device of claim 1 further comprising:
a convex peak of the adhesion geometry.

16. The ophthalmic medical device of claim 15 wherein the convex peak is a distance in a range of 0.001 to 0.1 millimeters.

17. The ophthalmic medical device of claim 1 wherein the ophthalmic medical device is manufactured from a material having a hardness in a range of 50 durometer Shore A to 80 durometer Shore A.

18. The ophthalmic medical device of claim 1 wherein the ophthalmic medical device is manufactured from a material having a tensile strength in a range of 6.0 to 9.0 MPa.

19. The ophthalmic medical device of claim 1 wherein the ophthalmic medical device is manufactured from a material having an elongation in a range of 180.0 to 220.0 percent.

20. The ophthalmic medical device of claim 1 wherein the ophthalmic medical device is manufactured from a material having a tear strength in a range of 12.0 to 19.0 kN/m.
